(19) United States
(12) Patent Application Publication
(10) Pub. No.: US 2007/0149853 A1
(43) Pub. Date: Jun. 28, 2007

(54) Industrial endoscope

(76) Inventor: Chun Yuan Chang, Taichung City (TW)

Correspondence Address: BANGER SHIA, 204 CANYON CREEK, VICTORIA, TX, 77901-3695, US

(21) Appl. No.: 11/317,552

(22) Filed: Dec. 24, 2005

(51) Int. Cl. A61B 1/00

(52) U.S. Cl. 600/146

(57) ABSTRACT

A lens-adjusting mechanism is disclosed for adjusting a lens of an industrial endoscope. The industrial endoscope further includes a cable connected to the lens. The lens-adjusting mechanism includes a telescopic structure for holding the cable, a lens holder for holding the lens and at least one connector for connecting the lens holder to the telescopic structure. Thus, the reduction of the length of the telescopic structure causes the bending of a section of the cable near the lens and the change of the view of the lens.

### BACKGROUND OF INVENTION

1. Field of Invention

The present invention relates to an accessory for an industrial endoscope and, more particular, to a mechanism for adjusting the lens of an industrial endoscope.

2. Related Prior Art

Referring to FIG. 1, a conventional endoscope includes a lens 1, a cable 2 and an eyepiece 3. The lens 1 and the eyepiece 3 are positioned at two ends of the cable 2. Views taken in the lens 1 can be transferred to the eyepiece 3 far away by means of optical elements such as optical fibers of the cable 2. Such an endoscope is often used to inspect parts or circuit boards located in a narrow and winding space in a machine to which access is not easy. The lens 1 can be advanced in such a narrow and winding space to an intended spot by means of the cable 2. Hence, inspection can be done without having to take the machine apart. Only when errors are spotted and repair is required will the machine be taken apart for further inspection and repair. Such an endoscope improves the convenience in such maintenance and inspection.

However, in such a narrow and winding space, the angle of the lens 1 is limited to the longitudinal direction of the most proximal section of the cable 2. To inspect a spot not in a forward sense of the longitudinal direction, a complicated system of refracting and reflecting lenses has to be used in the lens 1, or a delicate micro-machine has to be used to move the lens 1. Either way, a complicated and delicate arrangement is used, and precision is required. A high cost is inevitable. Moreover, a spot in a backward sense of the longitudinal direction cannot be inspected.

The present invention is therefore intended to obviate or at least alleviate the problems encountered in prior art.

### SUMMARY OF INVENTION

It is the primary objective of the present invention to provide an industrial endoscope with a mechanism for adjusting the lens of the industrial endoscope.

According to the present invention, a lens-adjusting mechanism is disclosed for adjusting a lens of an industrial endoscope. The industrial endoscope further includes a cable connected to the lens. The lens-adjusting mechanism includes a telescopic structure for holding the cable, a lens holder for holding the lens and at least one connector for connecting the lens holder to the telescopic structure. Thus, the reduction of the length of the telescopic structure causes the bending of a section of the cable near the lens and the change of the view of the lens.

Other objectives, advantages and features of the present invention will become apparent from the following description referring to the attached drawings.

### BRIEF DESCRIPTION OF DRAWINGS

The present invention will be described via detailed illustration of embodiments referring to the drawings.

FIG. 1 is a perspective view of a conventional industrial endoscope.

FIG. 2 is an exploded view of an endoscope according to the preferred embodiment of the present invention.

FIG. 3 is an enlarged partial view of the endoscope shown in FIG. 2.

FIG. 4 is another enlarged partial view of the endoscope of FIG. 2.

FIG. 5 is a cross-sectional partial view of the endoscope of FIG. 2.

FIG. 6 is another cross-sectional partial view of the endoscope shown in FIG. 2.

FIG. 7 is a side view of the endoscope shown in FIG. 2.

FIG. 8 is a side view of the endoscope in another position than shown in FIG. 7.

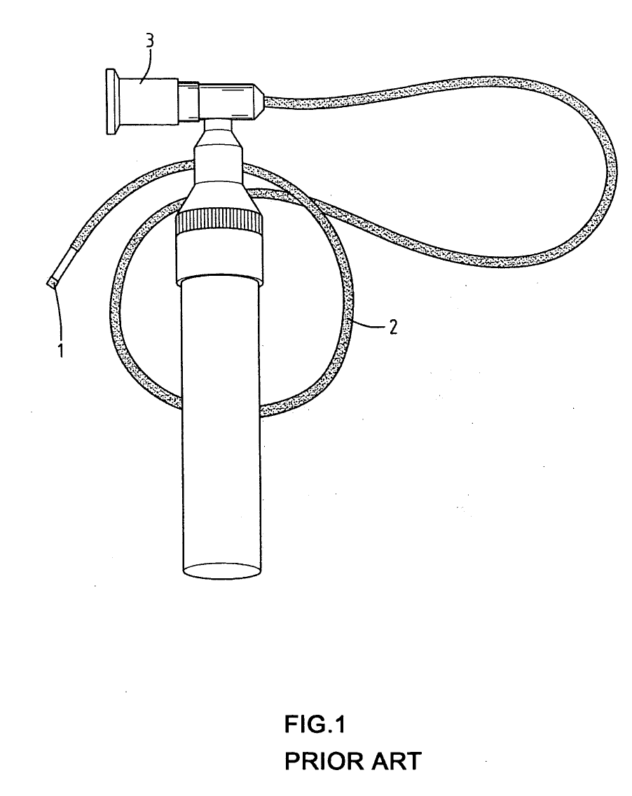

### DETAILED DESCRIPTION OF PREFERRED EMBODIMENT

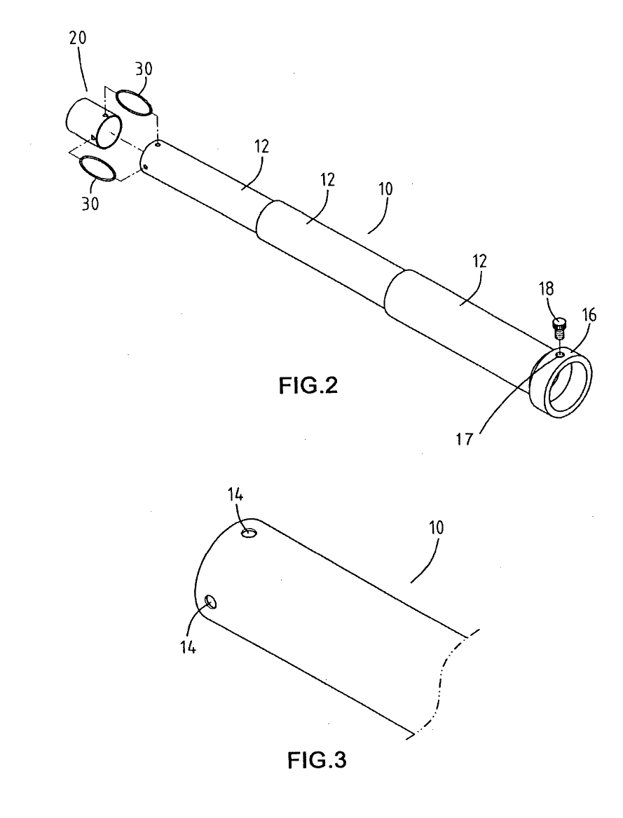

Referring to FIG. 2, there is shown a lens-adjusting device according to the preferred embodiment of the present invention. The mechanism includes a telescopic structure 10, a lens holder 20 and two connectors 30.

The telescopic structure 10 consists of three co-axial rigid pipes 12 so that the length of the telescopic structure 10 can be adjusted. For the convenience of the description, the rigid pipe 12 close to the lens holder 20 will be referred to as “first pipe.” The remaining rigid pipes 12 will be called “the second rigid pipe 12” and “the third rigid pipe 12” sequentially.

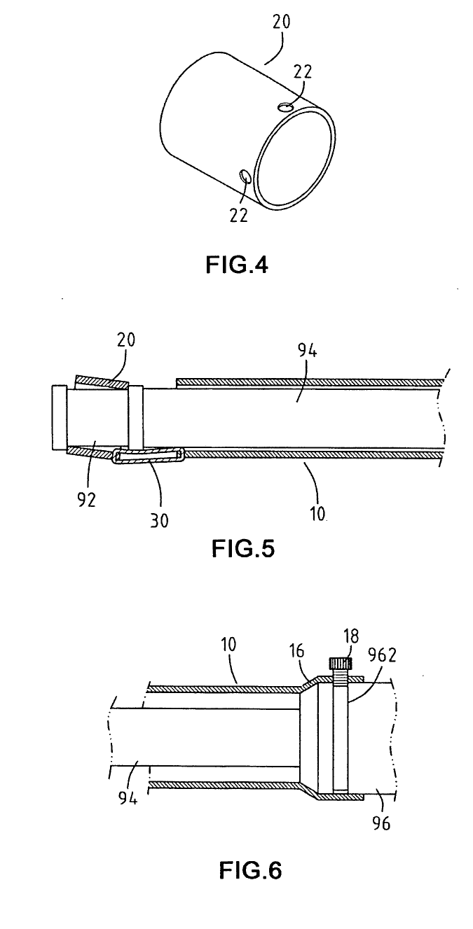

As best shown in FIG. 3, the first rigid pipe 12 defines two apertures 14. As best shown in FIG. 4, the lens holder 20 defines two apertures 22 corresponding to the apertures 14 of the first rigid pipe 12. Referring to FIG. 5, each of the connectors 30 is directed through related one of the apertures 14 and related one of the apertures 22 in order to connect the first rigid pipe 12 to the lens holder 20. The connectors 30 are loops in the preferred embodiment; however, they can be in any other proper form in another embodiment.

The third rigid pipe 12 is formed with an eyepiece holder 16. The eyepiece holder 16 defines a screw hole 17. A screw 18 is driven in the screw hole 17.

Referring to FIGS. 5 and 6, the lens-adjusting mechanism is provided around an endoscope in use. The endoscope includes a lens 92, a cable 94 and an eyepiece 96. A groove 962 is defined around the eyepiece 96.

Referring to FIG. 5, the lens holder 20 is provided around the lens 92 so that the movement of the former causes the movement of the latter. The lens holder 20 is preferably properly elastic for binding the lens 92.

Referring to FIG. 6, the eyepiece holder 16 is provided around the eyepiece 96. The screw 18 is driven into the groove 962 in order to bind the eyepiece holder 16 to the eyepiece 96.

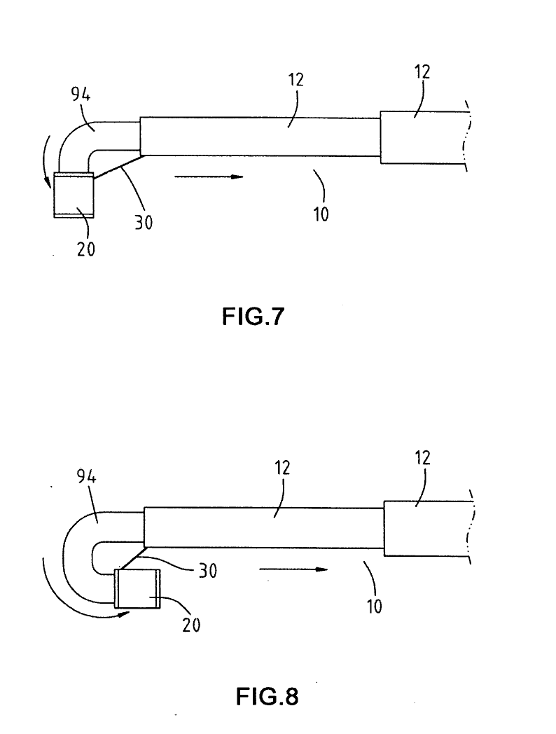

Referring to FIG. 7, to observe spots beside the telescopic structure 10, the rigid pipes 12 are moved on one another in order to reduce the length of the telescopic structure 10. There is exposed a section of the cable 94 near the lens 92. This section of the cable 94 is longer than the connectors 30. Thus, this section of the cable 94 is bent. Therefore, the axial direction of the lens 92 is perpendicular to the axial direction of the telescopic structure 10.

Referring to FIG. 8, to observe spots in a backward sense of the axial direction of the telescopic structure 10, the rigid pipes 12 are further moved on one another so as to further reduce the length of the telescopic structure 10. There is exposed a longer section of the cable 94 near the lens 92. This section of the cable 94 is much longer than the connectors 30. Thus, this section of the cable 94 is reversed. Therefore, the lens 92 can take views in backward sense of the axial direction of the telescopic structure 10.

With the lens-adjusting mechanism of the present invention including the telescopic structure 10, the lens holder 20 and the connectors 30, the easy adjustment of the length of the telescopic structure 10 causes the easy adjustment of the lens 92, without the need to use any complicated and delicate system of lenses or micro-mechanism.

The present invention has been described through the illustration of the preferred embodiment. Those skilled in the art can derive variations from the preferred embodiment without departing from the scope of the present invention. Therefore, the preferred embodiment shall not limit the scope of the present invention defined in the claims.

What is claimed is:

1. A lens-adjusting mechanism for adjusting a lens of an industrial endoscope, the lens-adjusting mechanism comprising:
a telescopic structure for holding a cable of the industrial endoscope;
a lens holder for holding the lens; and
at least one connector for connecting the lens holder to the telescopic structure so that the reduction of the length of the telescopic structure causes the bending of a section of the cable near the lens and the change of the view of the lens.

2. The lens-adjusting mechanism according to claim 1 wherein the connector is a loop.

3. The lens-adjusting mechanism according to claim 2 wherein the telescopic structure comprises an aperture for receiving the loop.

4. The lens-adjusting mechanism according to claim 2 wherein the lens holder comprises an aperture for receiving the loop.

5. The lens-adjusting mechanism according to claim 2 comprising two connectors.

6. The lens-adjusting mechanism according to claim 5 wherein the telescopic structure comprises two apertures for receiving the loops.

7. The lens-adjusting mechanism according to claim 5 wherein the lens holder comprises two apertures for receiving the loops.

8. The lens-adjusting mechanism according to claim 1 comprising an eyepiece holder connected to the telescopic structure in order to hold an eyepiece of the industrial endoscope.

9. The lens-adjusting mechanism according to claim 8 comprising a fastener for fastening the eyepiece holder to the eyepiece.

10. The lens-adjusting mechanism according to claim 9 wherein the fastener is a screw.

11. The lens-adjusting mechanism according to claim 10 wherein the eyepiece holder comprises a screw hole through which the screw is driven to the eyepiece.

12. The lens-adjusting mechanism according to claim 11 wherein the screw is driven into a groove defined in the eyepiece.
